(19) United States
(12) Patent Application Publication
(10) Pub. No.: US 2015/0160122 A1
(43) Pub. Date: Jun. 11, 2015

(54) DEVICE FOR MEASURING LIQUID PROPERTY AND USE THEREOF

(71) Applicant: AXALTA COATING SYSTEMS IP CO., LLC, Wilmington, DE (US)

(72) Inventors: Anthony Moy, Waxhaw, NC (US); Ayumu Yokoyama, Media, PA (US); Allan Blase Joseph Rodrigues, Bloomfield Hills, MI (US); Ken Stephen Schermacher, Chadds Ford, PA (US); Jeff B. Alspach, Lake Orion, MI (US)

(21) Appl. No.: 14/400,021

(22) PCT Filed: May 15, 2013
(86) PCT No.: PCT/US13/41045

§ 371 Date: Nov. 10, 2014

Related U.S. Application Data
(60) Provisional application No. 61/647,792, filed on May 16, 2012.

(51) Int. Cl. G01N 21/25; G01N 1/28; G01N 33/32

(52) U.S. Cl. CPC G01N 21/251; G01N 33/32; G01N 1/2813; G01N 2201/022

(57) ABSTRACT

The present disclosure is directed to a liquid measuring system for producing one or more property values of a liquid. The system can comprise: a thin film device and one or more measuring devices for measuring the one or more property values. This disclosure is particularly directed to a system comprise a thin film device for producing a thin film of the liquid on a spinning disk. The system is particularly useful for measuring color and appearance properties of the liquid. The system can be useful for producing coating compositions.

### CROSS-REFERENCE TO RELATED APPLICATIONS

This application is a U.S. National-Stage entry under 35 U.S.C. §371 based on International Application No. PCT/US2013/041044, filed May 15, 2013, which was published under PCT Article 21(2) and which claims priority to U.S. Provisional Application No. 61/647,792, filed May 16, 2012, and to International Application No. PCT/US2013/041045, filed May 15, 2013, which was published under PCT Article 21(2) and which claims priority to U.S. Provisional Application No. 61/647,800, filed May 16, 2012, which are all hereby incorporated in their entirety by reference.

### TECHNICAL FIELD

The present disclosure is directed to a thin film device and a liquid measuring system for producing one or more property values of a liquid. The disclosure is particularly directed to a thin film device and a system for measuring color and other properties of a wet thin film of a liquid.

### BACKGROUND

Compositions such as inks and coating compositions can be typically produced in liquid forms and can be produce into dry forms for industrial or consumer applications, such as printed materials or coatings on vehicles, appliances, or buildings. Production of such compositions can involve complex processes. The composition can have a plurality of properties including wet properties such as pH, viscosity, or wet color; and dry properties such as hardness or dry color. Typically, some of the wet properties can be different from the dry properties for the same composition. For example, wet color of a coating composition can be different from dry color of the same coating composition after cured or otherwise dried.

Currently, in order to produce a composition having desired dry properties, repeated tries and adjustments can be involved and can include the steps of producing an intermediate of the composition, drying it to form a dried composition, measuring dry properties of the dried composition, adjusting the composition, and repeating again until the desired properties are achieved. Such process is time and effort consuming and leads to time delays in production. For example, a coating composition batch can stay in a mixing tank for an extended time period while tests are run in control labs for producing dry samples to test coating properties.

Thus, needs exist for improved devices and processes to produce one or more property values of a liquid when it is wet. Furthermore, other desirable features and characteristics of the present invention will become apparent from the subsequent detailed description of the invention and the appended claims, taken in conjunction with the accompanying drawings and this background of the invention.

### SUMMARY

In accordance with an exemplary embodiment, a thin film device for producing a thin film of a liquid comprises:

a circular planar disk comprising a first surface and a second disk surface on the opposite sides of the circular planar disk, wherein the circular planar disk is coupled to a rotation shaft that is aligned with a rotational axis of the circular planar disk perpendicular to the disk surfaces for providing rotation to the circular planar disk along the rotational axis;

a device frame that positions the rotation shaft and the circular planar disk;

a thickness control device comprising a thin film setting edge coupled to a liquid return channel and at least one frame connector coupling the thin film setting edge and the liquid return channel to the device frame, wherein the frame connector is movable in respect to the device frame; and

a motion device coupled to the rotation shaft for providing rotation to the rotation shaft, and a motion control device for controlling rotation speed, rotation direction, or a combination thereof, of the motion device;

wherein the thickness control device is positioned at the first surface side of the circular planar disk, the thin film setting edge is substantially parallel to the first surface, and the thin film setting edge overlaps with the circular planar disk covering in a range of from about 50% to about 99% of the radius of the circular planar disk; and

wherein the distance between the thin film setting edge and the first surface is in a range of from about 0.05 mm to about 5 mm and adjustable via the frame connector.

In another exemplary embodiment, a liquid measuring system for producing one or more property values of a liquid is provided. The liquid measuring system comprises:

the thin film device disclosed herein; and
one or more measuring devices for measuring the one or more property values.

A kit for assembling into the thin film device also is disclosed herein.

### BRIEF DESCRIPTION OF DRAWINGS

The present invention will hereinafter be described in conjunction with the following drawing figures, wherein like numerals denote like elements, and:

FIGS. 1A and 1B show side cross-sectional schematic diagrams of examples of a thin film device: FIG. 1A is a cross-sectional view of a thin film device having a dipping reservoir and FIG. 1B is a cross-sectional view of a thin film device having a cup reservoir.

FIGS. 2A and 2B show side cross-sectional schematic diagrams of examples of a thin film device with liquid on a first surface: FIG. 2A is a cross-sectional view of a thin film device having a dipping reservoir and FIG. 2B is a cross-sectional view of a thin film device having a cup reservoir.

FIGS. 3A and 3B show frontal views of schematic diagrams of examples of the thin film device: FIG. 3A illustrates a thin film device having a dipping reservoir and FIG. 3B illustrates a thin film device having a cup reservoir.

FIG. 4A shows a side view of a schematic diagram of an example of a system having a frame connector configured vertically and FIG. 4B shows a side view of a schematic diagram of an example of a system having the frame connector configured horizontally.

FIGS. 5A and 5B show examples of configurations of measuring devices: FIG. 5A illustrates an example of a configuration for one or more measuring devices and FIG. 5B illustrates an example of a configuration for a device having one or more measuring functions.

FIG. 6 shows a top-down view of a schematic diagram of a part of an example of the thin film device.

FIG. 7 shows an example of a circular planar disk having a hiding pattern thereon.

FIG. 8 shows a diagram of an example of the system.

### DETAILED DESCRIPTION

The following detailed description is merely exemplary in nature and is not intended to limit the invention or the application and uses of the invention. Furthermore, there is no intention to be bound by any theory presented in the preceding background of the invention or the following detailed description.

The features and advantages of the present invention will be more readily understood, by those of ordinary skill in the art, from reading the following detailed description. It is to be appreciated that certain features of the invention, which are, for clarity, described above and below in the context of separate embodiments, may also be provided in combination in a single embodiment. Conversely, various features of the invention that are, for brevity, described in the context of a single embodiment, may also be provided separately or in any sub-combination. In addition, references in the singular may also include the plural (for example, “a” and “an” may refer to one, or one or more) unless the context specifically states otherwise.

The use of numerical values in the various ranges specified in this application, unless expressly indicated otherwise, are stated as approximations as though the minimum and maximum values within the stated ranges were both proceeded by the word “about.” In this manner, slight variations above and below the stated ranges can be used to achieve substantially the same results as values within the ranges. Also, the disclosure of these ranges is intended as a continuous range including every value between the minimum and maximum values.

A computing device used herein can refer to a data processing chip, a desktop computer, a laptop computer, a pocket PC, a personal digital assistant (PDA), a handheld electronic processing device, a smart phone that combines the functionality of a PDA and a mobile phone, or any other electronic devices that can process information automatically. A computing device can be built into other electronic devices, such as a built-in data processing chip integrated into an imaging device, color measuring device, or an appearance measuring device. A computing device can have one or more wired or wireless connections to a database, to another computing device, or a combination thereof. A computing device can be a client computer that communicates with a host computer in a multi-computer client-host system connected via a wired or wireless network including intranet and internet. A computing device can also be configured to be coupled with a data input or output device via wired or wireless connections. For example, a laptop computer can have data input devices such as key board, USB connections, or a touch screen and can be operatively configured to receive data and images through wired or wireless connections. A “portable computing device” can include a laptop computer, a pocket PC, a personal digital assistant (PDA), a handheld electronic processing device, a mobile phone, a smart phone, a tablet computer, or any other electronic devices that can process information and data and can be carried by a person.

Wired connections can include hardware couplings, splitters, adaptors, connectors, cables or wires. Wireless connections and devices can include, but are not limited to, Wi-Fi devices, Bluetooth devices, wide area network (WAN) wireless devices, local area network (LAN) devices, infrared communication devices, optical data transfer devices, radio transmitter and optionally receivers, wireless phones, wireless phone adaptor cards, or any other devices that can transmit signals in a wide range of radio frequency including visible or invisible optical wavelengths and electromagnetic wavelengths.

The term “wet” refers to a state of being liquid that is able to flow or adapt into a shape, such as the shape of a container or a substrate. Examples of wet compositions can include wet inks that have not dried or cured or wet coating compositions that have not dried or cured. A wet coating composition can be in a storage container or over a coated substrate and can adapt to the shape of the container or the shape of the substrate. A wet coating composition can comprise one or more liquid solvents, such as water, one or more organic solvents, one or more inorganic solvents, or a combination thereof. The “wet” property values refer to the property associated with composition values when the composition is being wet. A wet coating composition refers to a film or surface of the coating composition that is wet and not dry to the touch as determined by ASTM D1640.

The term “database” refers to a collection of related information that can be searched and retrieved. The database can be a searchable electronic numerical or textual document, a searchable PDF document, a Microsoft Excel® spreadsheet, an Microsoft Access® database (both supplied by Microsoft Corporation of Redmond, Wash.), an Oracle® database (supplied by Oracle Corporation of Redwood Shores, Calif.), or a Lynux database, each registered under their respective trademarks. The database can be a set of electronic documents, photographs, images, diagrams, or drawings, residing in one or more computer readable storage media that can be searched and retrieved. A database can be a single database or a set of related databases or a group of unrelated databases. “Related database” means that there is at least one common information element in the related databases that can be used to relate such databases. One example of the related databases can be Oracle® relational databases.

“Appearance” used herein refers to (1) the aspect of visual experience by which a coating or an object is viewed or recognized; and (2) perception in which the spectral and geometric aspects of a coating or an object is integrated with its illuminating and viewing environment. In general, appearance can include shape, texture, sparkle, glitter, gloss, transparency, color, opacity, other visual effects of a coating or an object, or a combination thereof. Appearance can vary with varying viewing angles or varying illumination angles.

Color data can be selected from or include L,a,b color values, L*,a*,b* color values, XYZ color values, L,C,h color values, spectral reflectance values, light absorption (K) and scattering (S) values (also known as “K,S values”), or a combination thereof, and can be stored in and retrieved from one or more databases. Other color values such as Hunter Lab color values, ANLAB color values, CIE LAB color values, CIE LUV color values, L*,C*,H* color values, any other color values known to or developed by those skilled in the art, or a combination thereof, can also be used.

This disclosure is directed to a thin film device (100) for producing a thin film (102) of a liquid. The thin film device can comprise:

a circular planar disk (101) comprising a first surface (101a) and a second disk surface (101b) on the opposite sides of the circular planar disk, the circular planar disk coupled to a rotation shaft (120) that is aligned with a rotational axis (110) of the circular planar disk perpendicular to the disk surfaces for providing rotation to the circular planar disk along the rotational axis (110);
a device frame (121) that positions the rotation shaft and the circular planar disk;
a thickness control device (125) comprising a thin film setting edge (125a) coupled to a liquid return channel (125b) and a frame connector (125c) coupling the thin film setting edge (125a) and the liquid return channel (125b) to the device frame (121), wherein the frame connector is movable in respect to the device frame; and
a motion device (130) coupled to the rotation shaft for providing rotation to the rotation shaft (120), and a motion control device (131) for controlling rotation speed, rotation direction, or a combination thereof, of the motion device;
wherein the thickness control device (125) is positioned at the first surface (101a) side of the circular planar disk (101), the thin film setting edge (125a) is substantially parallel to the first surface, and the thin film setting edge (125a) overlaps with the circular planar disk covering in a range of from about 50% to about 99% of the radius of the circular planar disk (101); and
wherein the distance (127) between the thin film setting edge (125a) and the first surface (101a) is in a range of from about 0.05 mm to about 5 mm and adjustable via the frame connector.

The thin film can be wet as defined above.

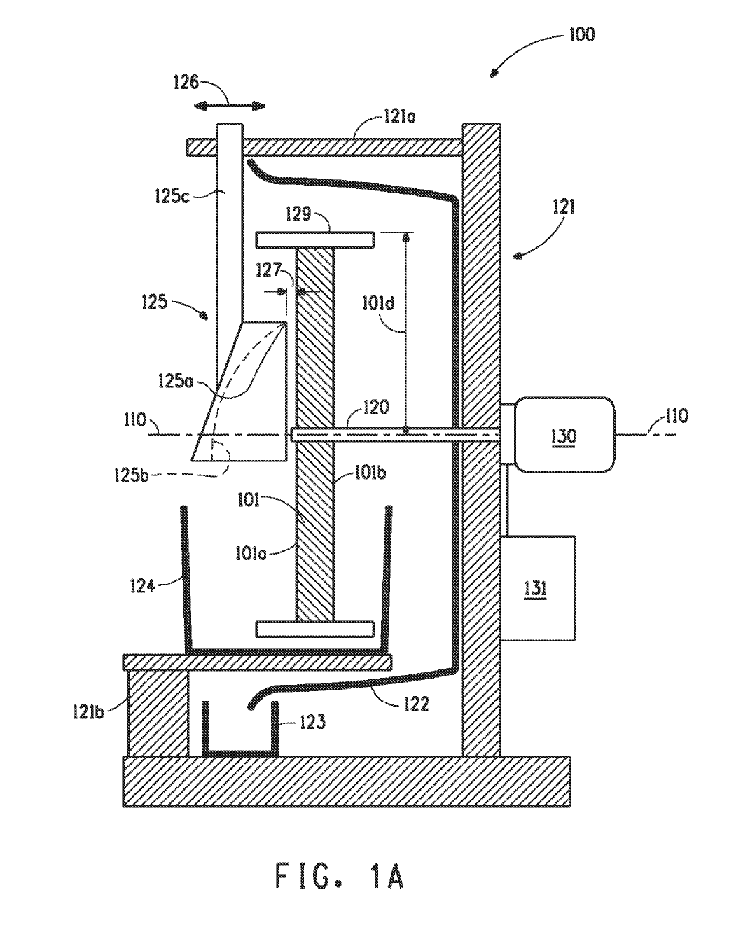

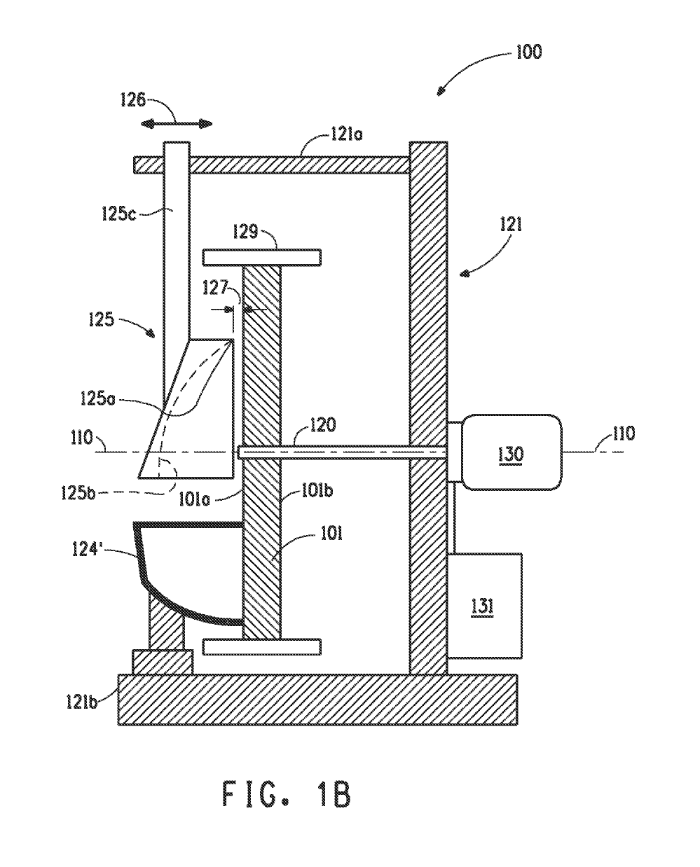

Examples of the thin film device can include the ones shown in FIG. 1A and FIG. 1B. The circular planar disk can be non-transparent or transparent.

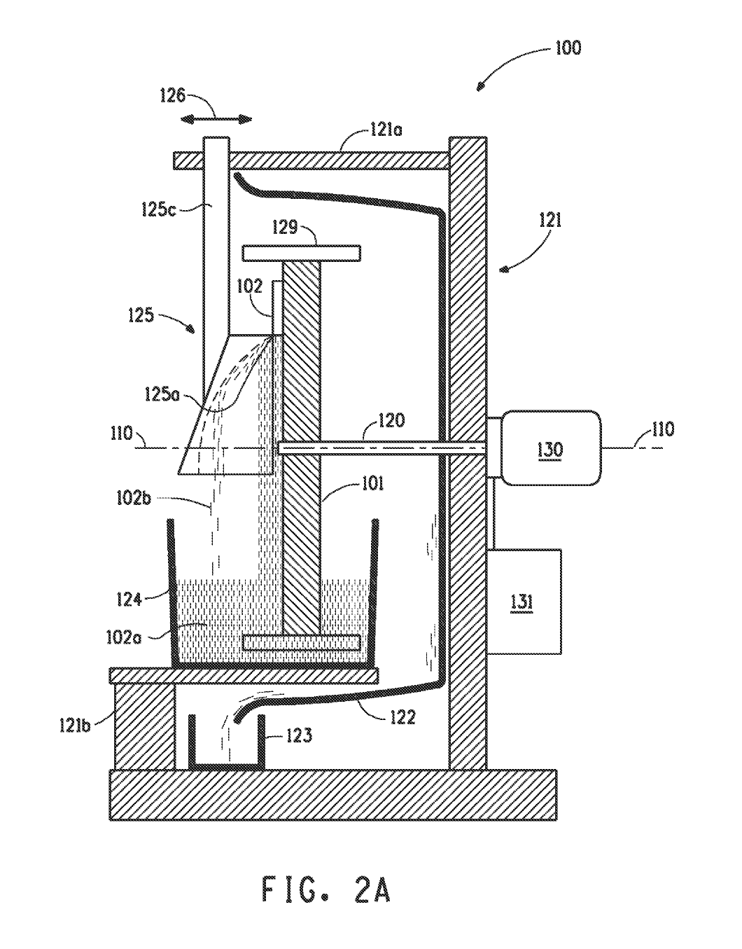

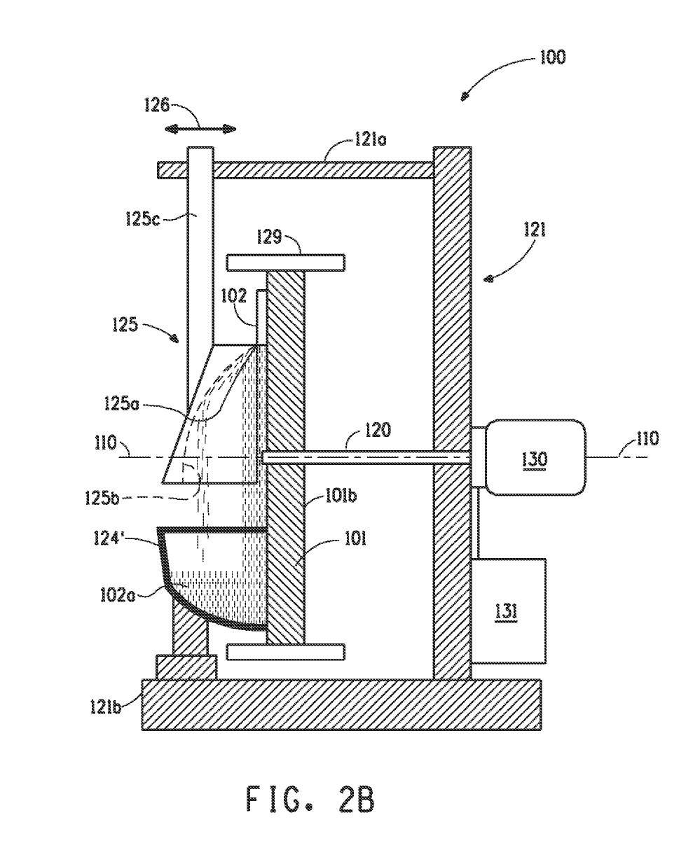

The thin film device can further comprise a first reservoir (124) for storing the liquid (102a) (FIG. 1A-1B and FIG. 2A-2B). The first reservoir can be configured so that the liquid, when present in the first reservoir, is in contact with at least a portion of the first surface. The first reservoir can be a dip reservoir (124), such as the one shown in FIG. 1A, or a cup reservoir (124′), such as the one shown in FIG. 1B. The dip reservoir can have a portion of the circular planar disk dipped within so the liquid, when present, can be in contact with the first surface and the second surface in the reservoir. The cup reservoir can provide the liquid to be in contact with only one of the surfaces, such as the first surface only.

The thin film device can further comprise a second reservoir (123) and a retainer (122). The second reservoir can be positioned to collect overflow of the liquid, when present, retained by the retainer. The retainer can be affixed to the device frame (121). The device frame (121) can have a frame base (121b) that can have one or more tiers for positioning the first and the second reservoirs.

The circular planar disk (101) can further comprise a circular retaining barrier (129) positioned at the circular edge of the circular planar disk. The circular retaining barrier can be a belt around the edge of the circular planar disk, a circular grove or a curved edge, a protruding around the edge of the disk, or a combination thereof

The circular planar disk (101) can be so positioned that the liquid, when present, is moved by the circular planar disk, when in rotation, from the first reservoir to the thin film setting edge against gravity. The first surface can be made of stainless steel, polymers, plastics, glass, or a combination thereof. The first surface should be suitable for forming a thin film of the liquid thereon having essentially even thickness for at least a portion of the first surface large enough for measuring properties of the liquid.

The thickness control device can be constructed from plastics, metals, glass, other suitable materials, or a combination thereof. Typically, all parts of the thin film device can be made from materials that are not reactive to the liquid. In one example, the thin film setting edge (125a) and the liquid return channel (125b) are molded from plastic materials and coupled to a frame connector (125c). In another example, the thin film setting edge and the liquid return channel can be constructed from the same or different materials and assembled together and then coupled to a frame connector. The liquid return channel can be configured to have a shape that allows the liquid to return to the first reservoir by gravity without disturbing the thin film formed. It is preferred that the liquid return channel is positioned below the thin film setting edge and above the first reservoir. The thin film setting edge and the liquid return channel can be coupled to one or more frame connectors. The one or more frame connectors can be coupled to the device frame via one or more frame couplings (121a) and can be moved in adjustment directions (126) to adjust the distance (127) between the thin film setting edge and the first surface. The thin film setting edge can be in a strait linear configuration, or a non-linear or curved configuration, as long as the distance between the thin film setting edge and the first surface is essentially the same along the edge and is in the range required herein so a thin film of the liquid having an essentially even thickness can be formed on the first surface. Being “essentially”, the distance between the thin film setting edge and the first surface can have small variations, typically less than about 20% variations along the edge. In one example, when a desired distance is about 0.5 mm, the actual distance can be, along the edge of the thin film setting edge, in a range of from about 0.4 mm to about 0.6 mm.

The length (125d) of the thin film setting edge (125a) can be in a range of from about 50% to about 99% of the radius (101d) of the circular planar disk. Typically, the thickness control device can be positioned within a projected space of the circular planar disk. The projected space is the space encircled by the imaginary boundary projected from the edge of the circular planar disk towards either sides of the circular planar disk parallel to the aforementioned rotational axis (110). The thickness control device can also be positioned beyond the projected space of the circular planar disk, especially when the circular planar disk is free from a protruding edge. In one example, the thickness control device is positioned so the thin film setting edge is positioned horizontally. In another example, the thickness control device is positioned so the thin film setting edge is positioned vertically. In yet another example, the thickness control device is positioned so the thin film setting edge is positioned at an angle between 0° (horizontally) and 90° (vertically).

This disclosure is also directed to a liquid measuring system for producing one or more property values of a liquid. The liquid measuring system can comprise:

the thin film device disclosed herein; and
one or more measuring devices for measuring the one or more property values.

Any of the aforementioned thin film devices are suitable for the liquid measuring system.

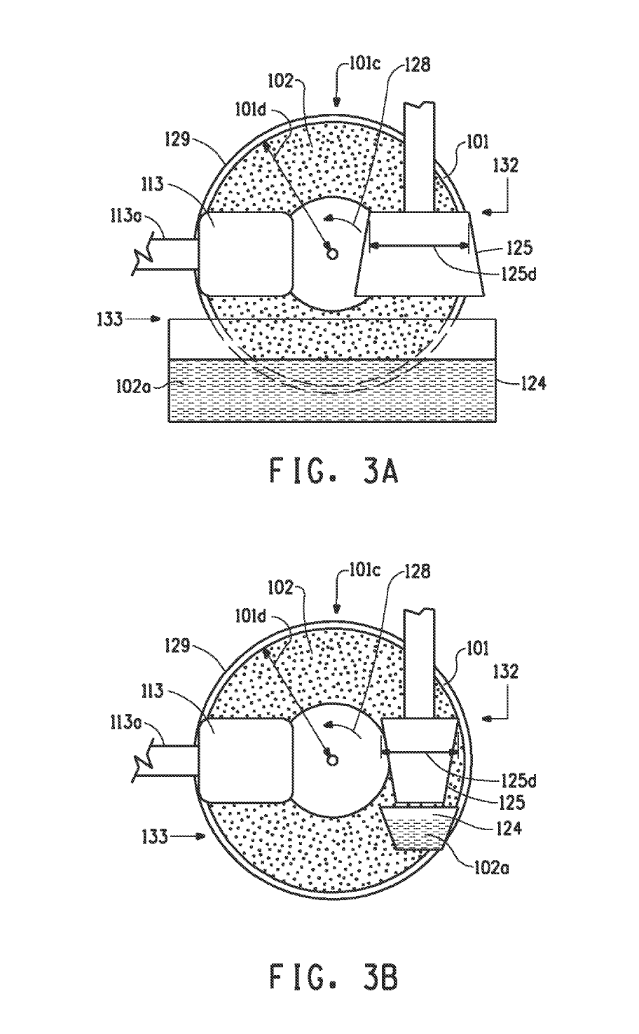

When in operation, the circular planar disk can be rotated by the motion device at a preset rotation speed and direction. The liquid in the first reservoir (102a) that is in contact with the circular planar disk can move along with the circular planar disk (FIG. 2A-2B). The surface of the circular planar disk can be selected based on the liquid to be measured so that the liquid can coat the surface evenly under the rotating conditions. The thin film setting edge can limit the amount or volume of the liquid that can pass the distance between the thin film setting edge and the first surface so a thin film (102) can be formed on the first surface. Excess liquid (102b) can be removed from the first surface by the thin film setting edge, collected and returned to the first reservoir (124 or 124′) by the liquid return channel by gravity. Any liquid that is spun off or splashed off the circular planar disk can be collected by the retainer (122) and returned to the second reservoir (123) (FIG. 2A), when present. The circular planar disk can be rotating at a measuring direction (128) (FIG. 3A-3B) such that the liquid is first going through a first point (132) that is aligned with the thin film setting edge, then through the top point (101c) of the circular planar disk, then through a second point (133) that is downstream of the top point (101c) and before the first reservoir.

The thin film formed in the area defined by the first point (132), the top point (101c) and the second point (133) can be suitable for measuring properties of the liquid and is herein referred to as “specimen measuring area”. With an appropriate rotation speed and amount of the liquid in the first reservoir, the thin film (102) in the specimen measuring area can be of essentially uniform thickness. By “essentially uniform thickness”, the thin film can have minor variations in thickness, such as in a range of from 0 to about 20%, percentage based on the average thickness of the thin film. Wet film thickness can be measured using methods or devices known to those skilled in the art.

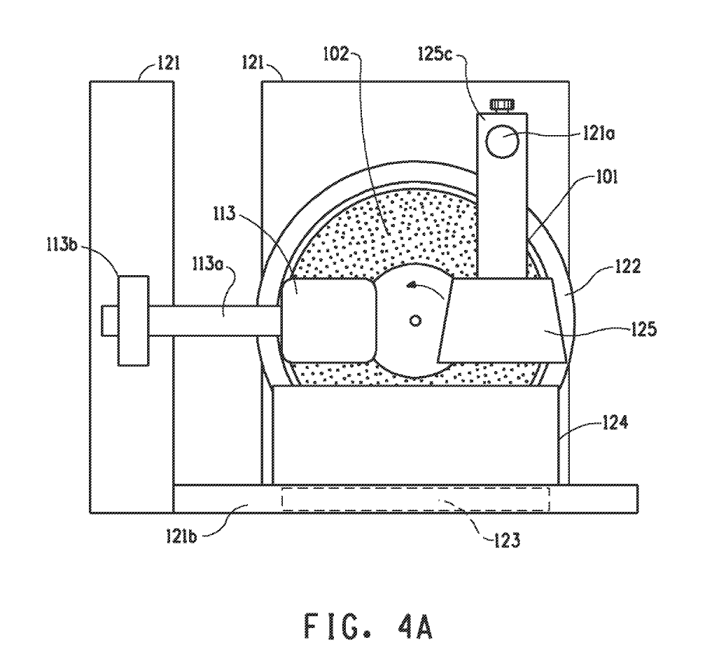

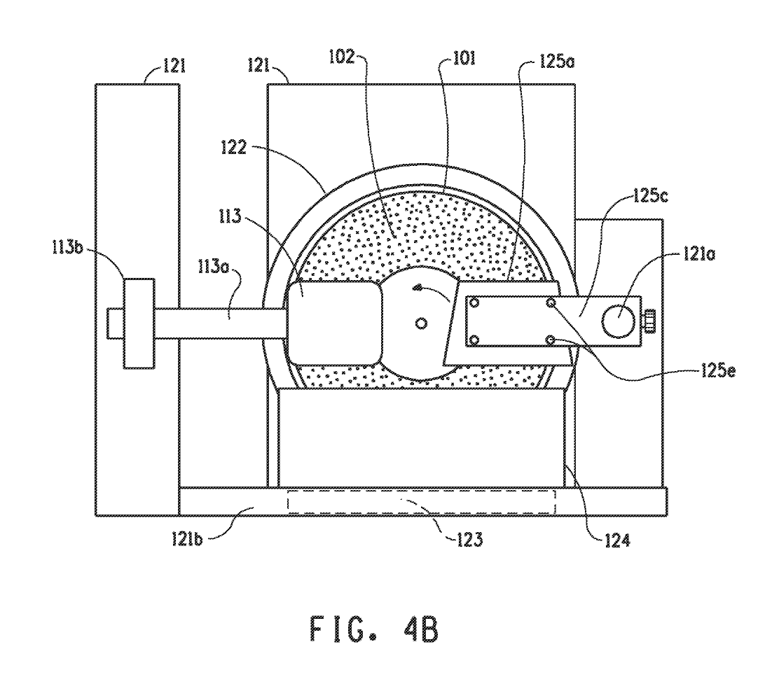

The one or more measuring devices can comprise a color measuring device (113) for measuring specimen color data of a specimen of the liquid, and optionally, an appearance measuring device for measuring specimen appearance data of the specimen. The one or more measuring devices can be coupled to the device frame with a measurement coupling (113a) that can be adjusted to position the measuring device at appropriate position and distance relative to the thin film, typically within the specimen measuring area. The measurement coupling (113a) can be coupled to the device frame via a measuring coupling (113b) (FIG. 4A-4B).

The thickness control device (125) can have the frame connector configured vertically (FIG. 4A) or horizontally (FIG. 4B). The thickness control device can further comprise one or more thickness adjusters (125e) that can adjust the distance between the thin film setting edge and the first surface, the position of the thin film setting edge, the angle of the thin film setting edge, the angle of the liquid return channel, or a combination thereof

The liquid measuring system can further comprise a film thickness measuring device for measuring the thickness of the thin film.

At least one of the measuring devices can comprise an illumination device (111) and a detection device (112) (FIG. 6), such as a light source, that can provide illumination at one or more pre-set illumination angles and detect reflection or spectrum at one or more pre-set viewing angles. A standard procedure can include ones described in ASTM E-2194, herein incorporated in by reference.

The one or more measuring devices can comprise a sparkle measuring device (141), a hiding measuring device (142), or a combination thereof. Other measuring devices that are determined suitable or developed by those skilled in the art for measuring the thin film can also be suitable.

The circular planar disk can have a hiding pattern affixed or embedded on the first surface. In one example, the circular planar disk can have a dark portion (150) and a light portion (151). In one example, a circular planar disk (101) having a black-and-white pattern, half black (150) and half white (151), can be used (FIG. 7). The measuring devices can take measurements synchronized with the motion device so measurements from the dark portion and the light portion can be recorded, respectively. In another example, hiding power of a coating composition can be measured using a color measuring device or a specific hiding measuring device to measure reflections of a thin film of the coating composition at the dark portion and the light portion, respectively. Hiding data value of the coating composition can be generated based on the ratio of the reflections at the dark and the light portions or a color difference measured over the black and white backgrounds. The color difference can be calculated using color difference calculation methods selected from ΔE, ΔE*_{ab}, ΔE*₉₄, or other color difference definitions or equations, such as the color differences (ΔE) based on BFD, CMC, CIE 1976, CIE 2000 (also referred to as CIEDE 2000), or any other color difference definitions or equations known to or developed by those skilled in the art. The hiding data can comprise film thickness data.

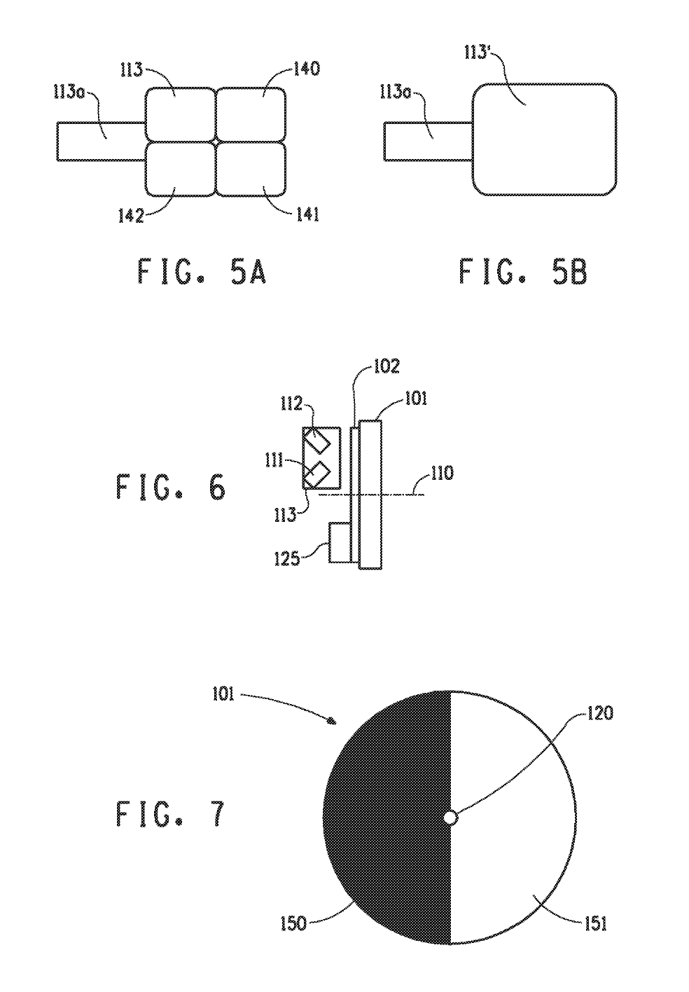

The one or more measuring devices can be configured to have individual measuring devices positioned in pre-determined locations or groupings. In one example, a color measuring device (113), an appearance measuring device (140), a sparkle measuring device (141) and a hiding measuring device (142) can be configured as a group (FIG. 5A). The one or more measuring devices can also be configured to have one single device (113′) with one or more measuring functions (FIG. 5B). Color measuring devices such as a colorimeter, a spectrophotometer, a goniospectrophotometer, or a combination thereof, can be suitable. Any suitable colorimeter or spectrophotometer, such as Model SP64 manufactured by X-Rite, Grandville, Mich., can be used. A goniospectrophotometer is also known as multi-angle spectrophotometer. Any suitable Goniospectrophotometers, such as Model MA68II from X-Rite, Grandville, Mich., or the ones provided by Murakami Color Research Laboratory, Tokyo, Japan, or by IsoColor Inc., Carlstadt, N.J., USA, can be used. Commercial instruments, such as BYK-mac available from BYK-Gardner USA, Columbia, Md., USA, that can measure color and sparkle, can also be suitable.

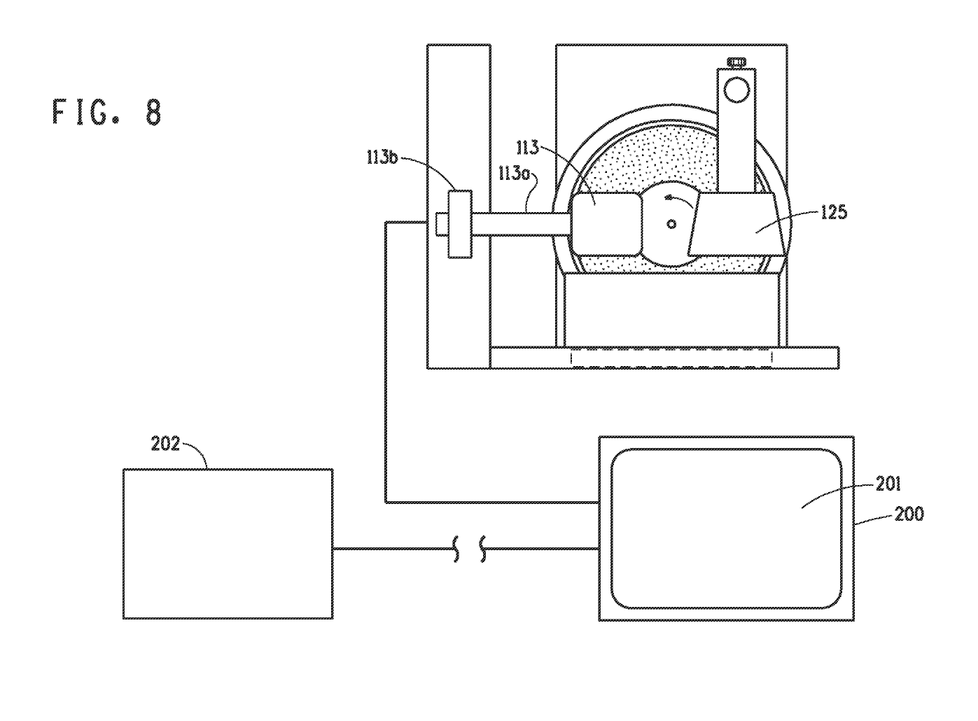

The liquid measuring system can further comprise a computing device (200) (FIG. 8) coupled to the color measuring device, optionally the motion device, and the appearance measuring device when present. The computing device can also be coupled to other measuring devices described above, such as the sparkle measuring device, the hiding measuring device, the film thickness measuring device, or a combination thereof. Any of the aforementioned computing devices can be suitable. A portable computing device is preferred.

The computing device can comprise a display device (201). Typical display devices, such a monitor, a TV, a high definition monitor, a touch screen, a HDR (high dynamic range) display, an LCD display screen, a plasma display screen, an LED display screen, a projector, a printer, or a combination thereof, are suitable. The computing device can comprise one or more display devices. The computing device and the display device can be a single device, such as a laptop computer or tablet computer, separate devices coupled via wired or wireless connections, such as a laptop with a wired or wireless display connections for example Push2TV™ from NETGEAR® under trademark or registered trademark from NETGEAR Inc., San Jose, Calif. 95134-1911, or a combination thereof

The liquid measuring system can further comprise a computing program product functionally coupled to the computing device. The computing program product can comprise computing program codes for:

receiving the specimen color data from the color measuring device;
optionally, receiving the specimen appearance data from the appearance measuring device when present;
generating specimen display data based on the specimen color data and optionally the specimen appearance data; and
displaying the specimen display data on the display device.

The computing program product can be installed on the computing device or installed on a network storage device and accessed from the computing device via wired or wireless connections. The computing program product can also be produced as a stand-alone product, such as a CD, DVD, a flash memory device comprising the computing program codes in a form that is readable or executable by a computing device.

The specimen display data can comprise R,G,B color data based on the specimen color data and optionally the specimen appearance data. Typically, R,G,B color data can be used for displaying digital image of colors or images on a digital display devices, such as aforementioned monitor, TV, high definition monitor, touch screen, HDR (high dynamic range) display, LCD display screen, plasma display screen, LED display screen, or projector. The specimen display data can be displayed as solid color images, realistic images, HDR (high dynamic range) images, realistic images rendered with BRDF (bidirectional reflectance distribution function), or a combination thereof, based on the specimen color data and optionally the specimen appearance data. The specimen display data can also be displayed as graphs, such as reflectance curves, spectral curves, numbers, or a combination thereof.

The liquid measuring system can further comprise a database (202). The database can be installed on the computing device, a data storage device accessible to the computing device, or a combination thereof. The database can be stored on a CD, DVD, flash memory device, a hard drive, a network drive, or a combination thereof. The database can be coupled to, or accessed from, the computing device via wired or wireless connections.

This disclosure is further directed to a system for producing a liquid composition. The system can comprise any of the aforementioned liquid measuring system. In one example, the liquid composition can be a coating composition and the system can be a coating production system.

This disclosure is further directed to a kit for assembling into a thin film device. The kit can comprise:

a circular planar disk (101) comprising a first surface (101a) and a second disk surface (101b) on the opposite sides of the circular planar disk, and a rotation shaft (120) connectable to the circular planar disk aligned with a rotational axis (110) of the circular planar disk perpendicular to the disk surfaces for providing rotation to the circular planar disk;
a device frame (121) connectable to the rotation shaft for positioning the circular planar disk and the rotation shaft;
a thickness control device (125) comprising a thin film setting edge (125a) coupled to a liquid return channel (125b) and at least one frame connector (125c) for coupling the thin film setting edge (125a) and the liquid return channel (125b) to the device frame (121), wherein the frame connector is connectable to the device frame and movable in respect to the device frame; and
a motion device (130) for providing rotation to the rotation shaft (120) and a motion control device (131) for controlling rotation speed, rotation direction, or a combination thereof, of the motion device, wherein the motion device is connectable to the rotation shaft;
when assembled, the thickness control device (125) and the first surface (101a) are positioned at the same side of the circular planar disk (101), the thin film setting edge (125a) is substantially parallel to the first surface, and the thin film setting edge (125a) overlaps with the circular planar disk covering in a range of from about 50% to about 99% of the radius of the circular planar disk (101); and the distance (127) between the thin film setting edge (125a) and the first surface (101a) is in a range of from about 0.05 mm to about 5 mm and adjustable via the frame connector.

The kit can further comprise a first reservoir (124) for storing the liquid. The kit can further comprise a second reservoir (123) and a retainer (122), wherein the second reservoir can be connectable to the device frame and can be so positioned to collect overflow of the liquid, when present, retained by the retainer, when assembled.

The circular planar disk (101) can further comprise a circular retaining barrier (129) positioned at the circular edge of the circular planar disk.

The kit can further comprise a color measuring device, an appearance measuring device, a sparkle measuring device, a hiding measuring device, or a combination thereof

The length (125d) of the thin film setting edge (125a) can be in a range of from about 50% to about 99% of the radius (101d) of the circular planar disk.

This disclosure is further directed to a process for producing one or more property values of a liquid. The process can comprise the steps of:

producing a thin film of a specimen of the liquid at a predetermined film thickness in a range of from about 0.05 mm to about 2 mm using a thin film device comprising:
a circular planar disk (101) comprising a first surface (101a) and a second disk surface (101b) on the opposite sides of the circular planar disk, the circular planar disk coupled to a rotation shaft (120) aligned with the rotational axis (110) of the circular planar disk perpendicular to the disk surfaces for providing rotation to the circular planar disk;
a device frame (121) that positions the circular planar disk and the rotation shaft;
a thickness control device (125) comprising a thin film setting edge (125a) coupled to a liquid return channel (125b) and at least one frame connector (125c) coupling the thin film setting edge (125a) and the liquid return channel (125b) to the device frame (121), the frame connector movable in respect to the device frame; and
a motion device (130) coupled to the rotation shaft for providing rotation to the rotation shaft (120), and a motion control device (131) for controlling rotation speed, rotation direction, or a combination thereof, of the motion device;
wherein the thickness control device (125) is positioned at the first surface (101a) side of the circular planar disk (101), the thin film setting edge (125a) is substantially parallel to the first surface, and the thin film setting edge (125a) overlaps with the circular planar disk covering in a range of from about 50% to about 99% of the radius of the circular planar disk (101);
wherein the distance (127) between the thin film setting edge (125a) and the first surface (101a) is in a range of from about 0.05 mm to about 5 mm and adjustable via the frame connector; and
wherein the predetermined film thickness is controlled by the distance (127) and the rotation speed and direction;
measuring the thin film with one or more measuring devices to produce specimen data; and producing the one or more property values based on the specimen data.

In the aforementioned process, the thin film can be produced by a thin film process comprising the steps of:

depositing the specimen of the liquid on a portion of the first surface; and
rotating the circular planar disk to form the thin film (102) on the first surface;
wherein the circular planar disk is rotating at a speed allowing the liquid to spread over the first surface, but not spinning off the circular planar disk.

The specimen can be deposited onto the first surface before the circular planar disk starts to spin or while it is spinning.

The thin film device can further comprise a first reservoir (124) for storing the liquid (102a), and at least a portion of the circular planar disk is in contact with the liquid stored in the first reservoir.

The circular planar disk can be rotating at a direction so that the liquid is moved by the circular planar disk from the first reservoir to the thin film setting edge against gravity to form the thin film on the first surface. Any overflow liquid, when present, can be removed from the first surface by the thin film setting edge, collected and returned to the first reservoir by the liquid return channel by gravity.

The one or more measuring devices comprise a color measuring device, and optionally, an appearance measuring device.

The one or more property values can comprise specimen color data and optionally specimen appearance data measured from the specimen of the liquid, the specimen color data can be selected from specimen color reflection data, specimen color spectral data, specimen color data derived from the specimen color reflection data, specimen color data derived from the specimen color spectral data, or a combination thereof. Property data for the wet liquid (wet data) and after the liquid is dry (dry data) can be converted, correlated, or otherwise related by testing, mathematical modeling, or a combination thereof. In one example, the one or more property values can comprise wet color data, dry color data derived from the wet color data, wet hiding data, dry hiding data derived from the wet hiding data, wet appearance data, dry appearance data derived from the wet appearance data, or a combination thereof

The process can further comprise the steps of:

generating from a computing device (200) specimen display data based on the specimen color data and optionally the specimen appearance data; and
displaying the specimen display data on a display device (201) coupled to the computing device.

The specimen display data can comprise R,G,B color data based on the specimen color data and optionally the specimen appearance data.

The color measuring device, optionally, the appearance measuring device, can be positioned at the first surface distal to the liquid returning channel for measuring the thin film on the first surface.

The process can further comprise the step of adjusting the distance between the color measuring device (113), optionally, the appearance measuring device (140), and the first surface.

The thin film device can further comprise a second reservoir (123) and a retainer (122). The second reservoir is positioned to collect overflow of the liquid, when present, retained by the retainer.

The circular planar disk (101) can further comprise a circular retaining barrier (129) positioned at the circular edge of the circular planar disk.

The length (125d) of the thin film setting edge (125a) can be in a range of from about 50% to about 99% of the radius (101d) of the circular planar disk.

The one or more measuring devices can comprise a sparkle measuring device (141), a hiding measuring device (142), or a combination thereof

The thin film thickness can be in a range of from about 0.05 mm to about 0.8 mm.

The process can further comprise the step of:

storing the one or more property values, the specimen data, or a combination thereof, in a database (202).

The specimen data can be produced by measuring the thin film with one or more measuring devices simultaneously or sequentially. The specimen data can be produced by measuring the thin film with one or more measuring devices at a same portion or different portions of the thin film. In one example, the color and appearance specimen data can be measured at the same portion of the thin film. In another example, the color specimen data, appearance specimen data, the hiding specimen data, can be measured at different portions of the thin film.

The liquid can be a coating composition.

The various embodiments herein can provide better film thickness control, especially for low viscosity liquid. In addition, the device can be easily cleaned since any liquid spun off or dripping off the disk can be collected.

Although the thin film device, the liquid measuring system and the process disclosed herein are specifically disclosed as suitable for producing coating compositions, they can be suitable for producing liquid that one or more properties are to be controlled or monitored. The liquid can include, for example, inks, dyes, beverages, or any other liquid for consumer use or industrial applications.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment, it being understood that various changes may be made in the function and arrangement of elements described in an exemplary embodiment without departing from the scope of the invention as set forth in the appended claims and their legal equivalents.

1. A thin film device for producing a thin film of a liquid, the thin film device comprising:
a circular planar disk comprising a first surface and a second disk surface on the opposite sides of the circular planar disk, the circular planar disk coupled to a rotation shaft that is aligned with a rotational axis of the circular planar disk perpendicular to the disk surfaces for providing rotation to the circular planar disk along the rotational axis;
a device frame that positions the rotation shaft and the circular planar disk;
a thickness control device comprising a thin film setting edge coupled to a liquid return channel and at least one frame connector coupling the thin film setting edge and the liquid return channel to the device frame, the frame connector movable in respect to the device frame; and
a motion device coupled to the rotation shaft for providing rotation to the rotation shaft, and a motion control device for controlling rotation speed, rotation direction, or a combination thereof, of the motion device;
wherein the thickness control device is positioned at the first surface side of the circular planar disk, the thin film setting edge is substantially parallel to the first surface, and the thin film setting edge overlaps with the circular planar disk covering in a range of from about 50% to about 99% of the radius of the circular planar disk; and
wherein the distance between the thin film setting edge and the first surface is in a range of from about 0.05 mm to about 5 mm and adjustable via the frame connector.

2. The thin film device of claim 1 further comprising a first reservoir for storing the liquid, wherein the first reservoir is configured so that the liquid, when present in the first reservoir, is in contact with at least a portion of the first surface.

3. The thin film device of claim 1 further comprising a second reservoir and a retainer, wherein the second reservoir is positioned to collect overflow of the liquid, when present, retained by the retainer.

4. The thin film device of claim 1, wherein the circular planar disk further comprises a circular retaining barrier positioned at the circular edge of the circular planar disk.

5. The thin film device of claim 1, wherein the circular planar disk is so positioned that the liquid, when present, is moved by the circular planar disk, when in rotation, from the first reservoir to the thin film setting edge against gravity.

6. The thin film device of claim 1, wherein the length of the thin film setting edge is in a range of from about 50% to about 99% of the radius of the circular planar disk.

7. A liquid measuring system for producing one or more property values of a liquid, the liquid measuring system comprising:
a circular planar disk comprising a first surface and a second disk surface on the opposite sides of the circular planar disk, the circular planar disk coupled to a rotation shaft that is aligned with a rotational axis of the circular planar disk perpendicular to the disk surfaces for providing rotation to the circular planar disk along the rotational axis;
a device frame that positions the rotation shaft and the circular planar disk;
a thickness control device comprising a thin film setting edge coupled to a liquid return channel and at least one frame connector coupling the thin film setting edge and the liquid return channel to the device frame, the frame connector movable in respect to the device frame; and
a motion device coupled to the rotation shaft for providing rotation to the rotation shaft, and a motion control device for controlling rotation speed, rotation direction, or a combination thereof, of the motion device;
wherein the thickness control device is positioned at the first surface side of the circular planar disk, the thin film setting edge is substantially parallel to the first surface, and the thin film setting edge overlaps with the circular planar disk covering in a range of from about 50% to about 99% of the radius of the circular planar disk; and
wherein the distance between the thin film setting edge and the first surface is in a range of from about 0.05 mm to about 5 mm and adjustable via the frame connector; and
one or more measuring devices for measuring the one or more property values.

8. The liquid measuring system of claim 7, wherein the thin film device further comprises a first reservoir for storing the liquid, the first reservoir configured so that the liquid, when present in the first reservoir, is in contact with at least a portion of the first surface.

9. The liquid measuring system of claim 7, wherein the thin film device further comprises a second reservoir and a retainer, the second reservoir positioned to collect overflow of the liquid, when present, retained by the retainer.

10. The liquid measuring system of claim 7, wherein the circular planar disk further comprises a circular retaining barrier positioned at the circular edge of the circular planar disk.

11. The liquid measuring system of claim 7, wherein the circular planar disk is so positioned that the liquid, when present, is moved by the circular planar disk, when in rotation, from the first reservoir to the thin film setting edge against gravity, and wherein the length of the thin film setting edge is in a range of from about 50% to about 99% of the radius of the circular planar disk.

12. The liquid measuring system of claim 7, wherein the one or more measuring devices comprise a color measuring device for measuring specimen color data of a specimen of the liquid, and optionally, an appearance measuring device for measuring specimen appearance data of the specimen.

13. The liquid measuring system of claim 12 further comprising a computing device coupled to the color measuring device, optionally the motion device, and the appearance measuring device when present.

14. The liquid measuring system of claim 13, wherein the computing device comprises a display device.

15. The liquid measuring system of claim 14 further comprising a computing program product functionally coupled to the computing device, wherein the computing program product comprises computing program codes for:
receiving the specimen color data from the color measuring device;
optionally, receiving the specimen appearance data from the appearance measuring device when present;
generating specimen display data based on the specimen color data and optionally the specimen appearance data; and
displaying the specimen display data on the display device.

16. The liquid measuring system of claim 15, wherein the specimen display data comprise R,G,B color data based on the specimen color data and optionally the specimen appearance data.

17. The liquid measuring system of claim 7, wherein the one or more measuring devices comprise a sparkle measuring device, a hiding measuring device, or a combination thereon.

18. (canceled)

19. (canceled)

20. (canceled)

21. (canceled)

22. (canceled)

23. (canceled)

24. (canceled)

25. (canceled)

26. A process for producing one or more property values of a liquid, the process comprising the steps of:
producing a thin film of a specimen of the liquid at a predetermined film thickness in a range of from about 0.05 mm to about 2 mm using a thin film device comprising:
a circular planar disk comprising a first surface and a second disk surface on the opposite sides of the circular planar disk, wherein the circular planar disk is coupled to a rotation shaft aligned with the rotational axis of the circular planar disk perpendicular to the disk surfaces for providing rotation to the circular planar disk;
a device frame that positions the circular planar disk and the rotation shaft;
a thickness control device comprising a thin film setting edge coupled to a liquid return channel and at least one frame connector coupling the thin film setting edge and the liquid return channel to the device frame, the frame connector is movable in respect to the device frame; and
a motion device coupled to the rotation shaft for providing rotation to the rotation shaft, and a motion control device for controlling rotation speed, rotation direction, or a combination thereof, of the motion device;
wherein the thickness control device is positioned at the first surface side of the circular planar disk, the thin film setting edge is substantially parallel to the first surface, and the thin film setting edge overlaps with the circular planar disk covering in a range of from about 50% to about 99% of the radius of the circular planar disk;
wherein the distance between the thin film setting edge and the first surface is in a range of from about 0.05 mm to about 5 mm and adjustable via the frame connector; and
wherein the predetermined film thickness is controlled by the distance and the rotation speed and direction;
measuring the thin film with one or more measuring devices to produce specimen data; and
producing the one or more property values based on the specimen data.

27. The process of claim 26, wherein the thin film is produced by a thin film process comprising the steps of:
depositing the specimen of the liquid on a portion of the first surface; and
rotating the circular planar disk to form the thin film on the first surface;
wherein the circular planar disk is rotating at a speed allowing the liquid to spread over the first surface, but not spinning off the circular planar disk.

28. The process of claim 26, wherein the thin film device further comprises a first reservoir for storing the liquid, and at least a portion of the circular planar disk is in contact with the liquid stored in the first reservoir.

29. The process of claim 26, wherein the circular planar disk is rotating at a direction so that the liquid is moved by the circular planar disk from the first reservoir to the thin film setting edge against gravity to form the think film on the first surface.

30. The process of claim 29, wherein any overflow liquid, when present, is removed from the first surface by the thin film setting edge, collected and returned to the first reservoir by the liquid return channel by gravity.

31. The process of claim 26, wherein the one or more measuring devices comprise a color measuring device, and optionally, an appearance measuring device.

32. The process of claim 31, wherein the one or more property values comprise specimen color data and optionally specimen appearance data measured from the specimen of the liquid, the specimen color data are selected from specimen color reflection data, specimen color spectral data, specimen color data derived from the specimen color reflection data, specimen color data derived from the specimen color spectral data, or a combination thereof.

33. The process of claim 32 further comprising the steps of:
generating from a computing device specimen display data based on the specimen color data and optionally the specimen appearance data; and
displaying the specimen display data on a display device coupled to the computing device.

34. The process of claim 33, wherein the specimen display data comprise R,G,B color data based on the specimen color data and optionally the specimen appearance data.

35. The process of claim 31, wherein the color measuring device, optionally, the appearance measuring device, is positioned at the first surface distal to the liquid returning channel for measuring the thin film on the first surface.

36. The process of claim 35 further comprising the step of adjusting the distance between the color measuring device, optionally, the appearance measuring device, and the first surface.

37. The process of claim 26, wherein the thin film device further comprises a second reservoir and a retainer, the second reservoir is positioned to collect overflow of the liquid, when present, retained by the retainer.

38. The process of claim 26, wherein the circular planar disk further comprises a circular retaining barrier positioned at the circular edge of the circular planar disk.

39. The process of claim 26, wherein the length of the thin film setting edge is in a range of from about 50% to about 99% of the radius of the circular planar disk.

40. The process of claim 26, wherein the one or more measuring devices comprise a sparkle measuring device, a hiding measuring device, or a combination thereof.

41. The process of claim 26, wherein the thin film thickness is in a range of from about 0.05 mm to about 0.8 mm.

42. The process of claim 26, further comprising the step of:
storing the one or more property values, the specimen data, or a combination thereof, in a database.

43. The process of claim 26, wherein the specimen data are produced by measuring the thin film with one or more measuring devices simultaneously or sequentially.

44. The process of claim 26, wherein the specimen data are produced by measuring the thin film with one or more measuring devices at a same portion or different portions of the thin film.

45. The process of claim 26, wherein the liquid is a coating composition.
